(19) United States
(12) Patent Application Publication
(10) Pub. No.: US 2026/0088163 A1
(43) Pub. Date: Mar. 26, 2026

(54) SIMULATED PERIPHERAL CONNECTIONS TO CAPITAL EQUIPMENT

(71) Applicant: Boston Scientific Scimed, Inc., Maple Grove, MN (US)

(72) Inventors: Steven Kinio, Pickering (CA); Foss Jiao, Vaughan (CA); Deep Patel, Waterdown (CA); Gareth Davies, Toronto (CA)

(21) Appl. No.: 19/339,114

(22) Filed: Sep. 24, 2025

Related U.S. Application Data
(60) Provisional application No. 63/698,811, filed on Sep. 25, 2024.

(51) Int. Cl. G16H 40/63; A61B 17/00; A61B 18/00; A61B 18/14; A61B 90/98; G06F 21/44

(52) U.S. Cl. CPC G16H 40/63; A61B 18/1492; A61B 90/98; G06F 21/44; A61B 2017/0023; A61B 2017/00486; A61B 2018/00577

(57) ABSTRACT

A system for performing a medical procedure using a piece of capital equipment includes a peripheral device adapter. The adapter includes a housing having a first end configured to connect to the piece of capital equipment and a second end configured to connect to a single use device. The adapter includes a connector configured to removably connect to a peripheral device. The adapter includes a peripheral device adapter memory device for storing instructions, wherein the instructions are configured to package data or information associated with the peripheral device into a format representative of a single use device to allow for communication with the piece of capital equipment. The system includes one or more processors. The one or more processors is configured for detecting a peripheral device connected to the peripheral device adapter when the peripheral device adapter is connected to the piece of capital equipment; querying the detected connected peripheral device; and receiving a response from the connected peripheral device and authenticating the connected peripheral device.

### CROSS REFERENCE TO RELATED APPLICATIONS

The present applications claims priority to U.S. Provisional Patent Application No. 63/698,811 filed Sep. 25, 2024, the disclosure of which is incorporated herein in its entirety.

### TECHNICAL FIELD

The present disclosure relates to medical systems and methods for providing treatment to a tissue. More specifically, the present disclosure relates to devices and methods allowing communication between capital equipment, peripheral devices, and single use devices.

### BACKGROUND

Capital equipment is not consumed in the normal course of business and often times has long service lives. Capital equipment systems in the medical field can include control systems, RF generators, imaging systems, diagnostic equipment, monitors, and other systems. It is challenging to design capital equipment that is capable of interfacing with future, unknown peripherals, especially from a hardware-design perspective. Using USB connections on capital equipment for future peripherals allows for connection but leads to cybersecurity risks, and use up valuable hardware and driver space on the PCB and in the firmware/software. A better solution involves leveraging the communication system used by generators for reading single-use-devices (SUDs) to allow arbitrary peripherals to share data with the system, with no additional connectors required.

### SUMMARY

The proposed invention leverages the communication system used by generators or other capital equipment for reading single use devices (SUDs) to allow arbitrary peripherals to share data with the system, with no additional connectors required.

Example 1 is a system for performing a medical procedure using a piece of capital equipment. The system includes a peripheral device adapter. The adapter includes a housing having a first end configured to connect to the piece of capital equipment and a second end configured to connect to a single use device. The adapter includes a connector configured to removably connect to a peripheral device. The adapter includes a peripheral device adapter memory device for storing instructions, wherein the instructions are configured to package data or information associated with the peripheral device into a format representative of a single use device to allow for communication with the piece of capital equipment.

Example 2 is the system of Example 1, wherein the peripheral device includes one or more sensor, therapy controller, monitor, or therapy device.

Example 3 is the system of any of Examples 1 or 2, wherein the piece of capital equipment includes a control system, power or energy generator, ablation system, therapy controller, or console.

Example 4 is the system of any of Examples 1-3, wherein the single use device includes one or more lead, perforation device, electrode, catheter, sheath, guidewire, or dilator.

Example 5 is the system of any of Examples 1-4, wherein energy is delivered through the peripheral device adapter to one or more electrodes of the single use device connected to the second end of the peripheral device adapter in order to perforate tissue during a transseptal crossing procedure.

Example 6 is the system of any of Examples 1-4, wherein energy is delivered through the peripheral device adapter to one or more electrodes of the single use device connected to the second end of the peripheral device adapter in order to ablate tissue.

Example 7 is the system of any of Examples 1-6, wherein the peripheral device adapter is configured for 1-wire, I2C, CAN, parallel-bus architecture, or low-level data communication protocol.

Example 8 is the system of any of Examples 1-7, wherein the format representative of a single use device allows the peripheral device to appear as a single use device during communication with the piece of capital equipment.

Example 9 is the system of any of Examples 1-8, wherein the peripheral device adapter memory includes eeprom, flash, SD, MMC, XD, or SDHC.

Example 10 is the system of any of Examples 1-9, wherein the peripheral deice adapter memory is removable.

Example 11 is the system of any of Examples 1-10, wherein the peripheral device adapter is configured to connect to an additional peripheral device adapter.

Example 12 is the system of any of Examples 1-11, wherein the second end configured to connect to a single use device includes an over the wire connector.

Example 13 is the system of any of Examples 1-12, wherein the peripheral device adapter allows for signals to pass from the piece of capital equipment to the single use device.

Example 14 is the system of Example 13, wherein the signals include energy for delivery to a patient for therapy.

Example 15 is the system of Examples 1-14, further comprising one or more processors. The one or more processors is configured for detecting a peripheral device connected to the peripheral device adapter when the peripheral device adapter is connected to the piece of capital equipment; querying the detected connected peripheral device; receiving a response from the connected peripheral device; authenticating the connected peripheral device; unlocking one or more pages in the peripheral device adapter memory; and transmitting information between the peripheral device and the piece of capital equipment.

Example 16 is a system for performing a medical procedure using a piece of capital equipment. The system includes a peripheral device adapter. The adapter includes a housing having a first end configured to connect to the piece of capital equipment and a second end configured to connect to a single use device. The adapter includes a connector configured to removably connect to a peripheral device. The adapter includes a peripheral device adapter memory device for storing instructions, wherein the instructions are configured to package data or information associated with the peripheral device into a format representative of a single use device to allow for communication with the piece of capital equipment. The system includes one or more processors. The one or more processors is configured for detecting a peripheral device connected to the peripheral device adapter when the peripheral device adapter is connected to the piece of capital equipment. The one or more processors is configured for querying the detected connected peripheral device. The one or more processors is configured for receiving a response from the connected peripheral device and authenticating the connected peripheral device.

Example 17 is the system of Example 16, wherein the peripheral device includes one or more sensor, therapy controller, monitor, or therapy device.

Example 18 is the system of Example 16, wherein the piece of capital equipment includes a control system, power or energy generator, ablation system, therapy controller, or console.

Example 19 is the system of Example 16, wherein the single use device includes one or more lead, perforation device, electrode, catheter, sheath, guidewire, or dilator.

Example 20 is the system of Example 16, wherein energy is delivered through the peripheral device adapter to one or more electrodes of the single use device connected to the second end of the peripheral device adapter in order to perforate tissue during a transseptal crossing procedure.

Example 21 is the system of Example 16, wherein energy is delivered through the peripheral device adapter to one or more electrodes of the single use device connected to the second end of the peripheral device adapter in order to ablate tissue.

Example 22 is the system of Example 16, wherein the peripheral device adapter is configured for 1-wire, I2C, CAN, parallel-bus architecture, or low-level data communication protocol.

Example 23 is the system of Example 16, wherein the peripheral device adapter memory is removable.

Example 24 is the system of Example 16, wherein the peripheral device adapter memory includes eeprom, flash, SD, MMC, XD, or SDHC.

Example 25 is the system of Example 16, wherein the peripheral device adapter is configured to connect to an additional peripheral device adapter.

Example 26 is the system of Example 16, wherein the second end configured to connect to a single use device includes an over the wire connector.

Example 27 is the system of Example 16, wherein the peripheral device adapter allows for energy to pass from the piece of capital equipment to the single use device for delivery to a patient for therapy.

Example 28 is the system of Example 16, wherein the one or more processors is further configured for unlocking one or more pages in the peripheral device adapter memory; and transmitting information between the peripheral device and the piece of capital equipment.

Example 29 is a method for communicating between a peripheral device and a piece of capital equipment. The method includes detecting the peripheral device connected to the piece of capital equipment, wherein the peripheral device includes data or information packaged into a format representative of a single use device. The method includes querying the peripheral device and receiving a response from the peripheral device. The method includes authenticating the peripheral device. The method includes unlocking one or more pages in a memory of the peripheral device and transmitting information between the connected peripheral device and the piece of capital equipment.

Example 30 is the method of Example 29, wherein transmitting information between the connected peripheral device and the piece of capital equipment includes receiving information from the connected peripheral device and sending information to the connected peripheral device.

Example 31 is the method of Example 29, wherein the one or more pages includes access to data associated with the connected peripheral device.

Example 32 is the method of Example 29, wherein contents of the one or more pages can be updated dynamically.

Example 33 is the method of Example 29, further comprising electrically connecting a single use device to the piece of capital equipment.

Example 34 is the method of Example 33, further comprising sending signals from the piece of capital equipment to the single use device though an adapter electrically connecting the peripheral device, the capital equipment, and the single use device.

Example 35 is the method of Example 34, further comprising identifying data indicating activation of a switch on the single use device and modifying the signals.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the disclosure.

FIG. 2 is an exploded schematic representation of a system for performing a medical procedure and allowing communication between peripheral devices, capital equipment, and single use devices, in accordance with an embodiment of the disclosure.

FIG. 3 is a schematic representation of a system for performing a medical procedure and for allowing communication between peripheral devices, capital equipment, and single use devices having a single peripheral device adapter, in accordance with an embodiment of the disclosure.

FIG. 4 is a schematic representation of a system for performing a medical procedure and for allowing communication between peripheral devices, capital equipment, and single use devices having a plurality of peripheral device adapters, in accordance with an embodiment of the disclosure.

FIG. 5 is a schematic illustration of a computing platform associated with capital equipment for controlling communication between peripheral devices, capital equipment, and single use devices, in accordance with an embodiment of the disclosure.

FIG. 6 is a flowchart illustration a method for providing therapy to a patient using a single-use RF device, in accordance with an embodiment of the disclosure.

FIG. 7 illustrates one or more simulated pages located in a peripheral device adapter memory, in accordance with an embodiment of the disclosure.

FIG. 8 is a schematic representation of a single use device connector having a push button switch, in accordance with an embodiment of the disclosure.

FIG. 9 illustrates an expansion block for use with a system for performing a medical procedure and allowing communication between peripheral devices, capital equipment, and single use devices, in accordance with an embodiment of the disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

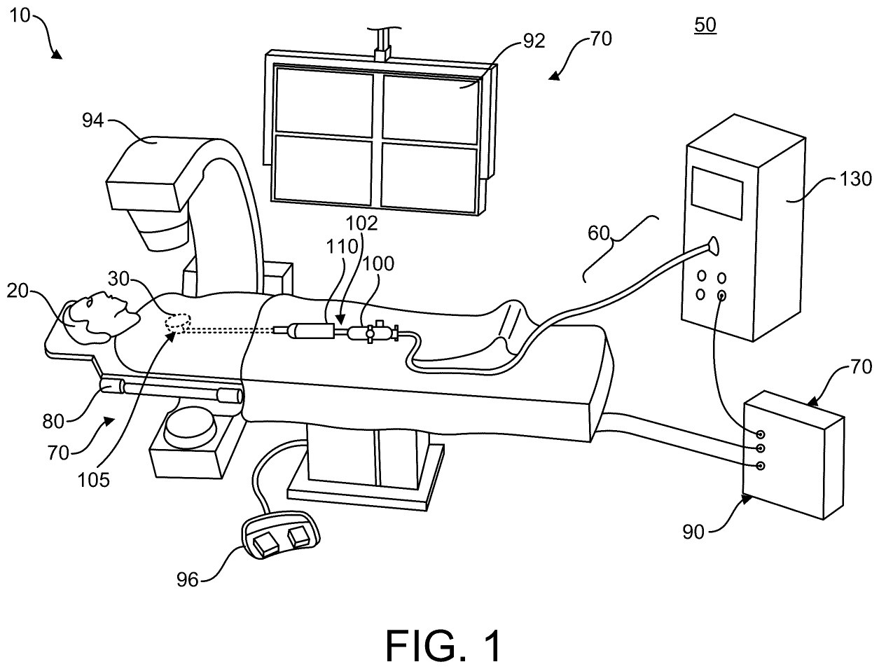

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. Exemplary procedures include, without limitation, deployment of diagnostic or mapping catheters within a left atrium for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures are merely illustrative and in no way limiting with respect to the present disclosure.

The electrophysiology system 50 includes an access system 60 and an EAM system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The access system 60 includes a dilator 100 having a proximal portion 102 and a distal portion 105, an introducer sheath 110, and a console 130. In some aspects, the distal portion 105 includes a tapered region. Additionally, the access system 60 includes various connecting elements, e.g., cables, umbilicals, and the like, that operate to functionally connect the components of the access system 60 to one another and to the components of the EAM system 70.

In some embodiments, the introducer sheath 110 is operable to provide a delivery conduit through which the dilator 100, in particular all or part of the distal portion 105 thereof, can be deployed to the specific target sites within the patient's heart 30. In some aspects, the sheath 110 provides a delivery conduit for medical devices other than the dilator 100. The dilator 100 is configured with a lumen such that a guiding device, for example a guide wire, or perforation device, for example an RF perforation device, can be inserted therein. In some aspects, a perforation device can be used to perform a transseptal crossing procedure within the heart 30. It is understood that different workflows may necessitate different order of operations for positioning of components, for example the introducer sheath, 110, dilator 100, and perforation device inside the body at any given time. For example, in some procedures the dilator 100 may or may not be present during access to the patent's heart 30.

The console 130 is configured to control functional aspects of the access system 60. In embodiments, the console 130 includes one or more controllers, microprocessors, and/or computers that execute code out of memory to control and/or perform the functional aspects of the access system 60. In embodiments, the memory can be part of the one or more controllers, microprocessors, and/or computers, and/or part of memory capacity accessible through a network, such as the world wide web. In embodiments, the console 130 can include pulse generator hardware, software and/or firmware configure to generate electrical pulses in predefined waveforms, which can be transmitted to electrodes positioned on the dilator 100, guiding device, or perforation device to generate electric fields sufficient to achieve the desired clinical effect, for example ablation of target tissue through irreversible electroporation. In embodiments, the console 130 can deliver the pulsed waveforms in a monopolar or bipolar mode of operation.

The EAM system 70 is operable to track the location of the various functional components of the access system 60, and to generate high-fidelity three-dimensional anatomical and electro-anatomical maps of the cardiac chambers of interest. In embodiments, the EAM system 70 can be the OPAL™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web. As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

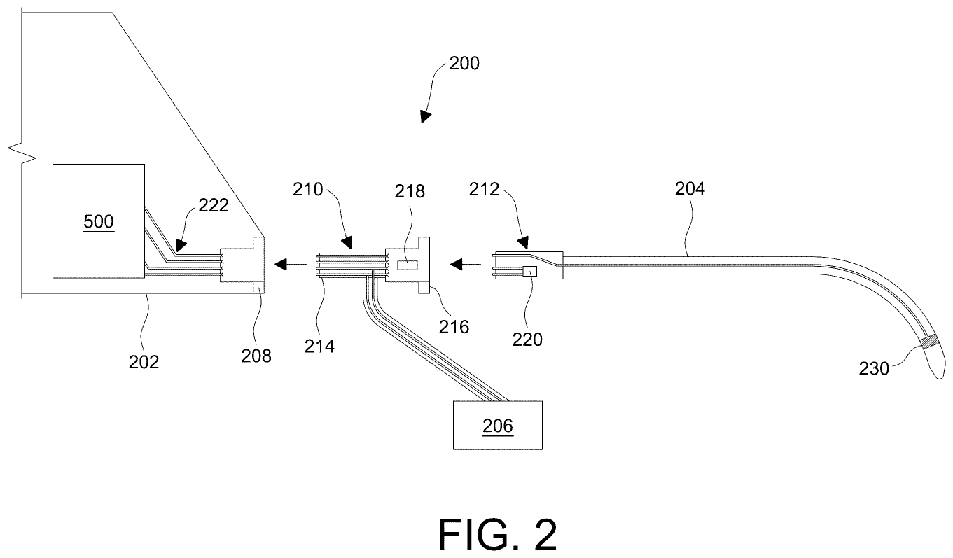

FIG. 2 is an exploded schematic representation of a system 200 for performing a medical procedure and for allowing communication between peripheral devices, capital equipment, and single use devices, in accordance with an embodiment of the disclosure. The system 200 includes a piece of capital equipment 202, for example a control system, power or energy generator, ablation system, therapy controller, console 130, EAM system 70, or other piece of equipment configured for repeated use and connection with other devices to control, direct, or otherwise ensure operation during a medical procedure. The capital equipment 202 is configured to connect to a single use device 204 for use during a medical procedure. For example, the single use device 204 may include one or more lead, perforation device, electrode, catheter, sheath 110, guidewire, or dilator 100. The single use device 204 can include one or more electrodes 230 configured to deliver energy to a target tissue during a medical procedure, such as during an ablation or cauterization procedure, or provide physiological information about a patient to the capital equipment. The capital equipment 202 is configured to deliver signals to the single use device 204. The signals may include energy for delivery to a patient for therapy, such as during an ablation or cauterization procedure.

The system 200 includes a peripheral device 206 that is connectable to the capital equipment 202 as well as the single use device 204. The peripheral device 206 is any device capable of providing information to the capital equipment 202 or from the capital equipment 202 during a medical procedure. In one aspect, the peripheral device 206 may include one or more sensor, therapy controller, monitor, or therapy device that can receive information or signals from the capital equipment 202 or deliver information or signals regarding the medical environment, such as physiological information, to the capital equipment 202.

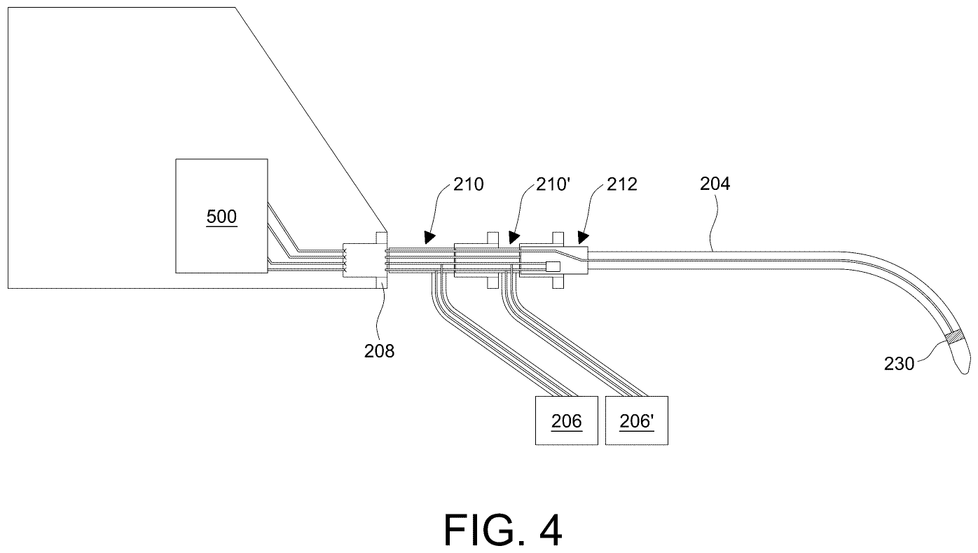

The capital equipment 202 includes a capital equipment connector 208. The capital equipment connector 208 is configured to mechanically and electrically connect with a peripheral device adapter 210 of the peripheral device 206 and a single use device connector 212 of the single use device 204. The capital equipment connector 208 is configured to receive a peripheral device adapter 210 or a single use device connector 212. In medical procedures where only a single use device 204 is required, the single use device connector 212 is inserted into the capital equipment connector 208 to electrically connect the single use device 204 to the capital equipment 202. In medical procedures where in addition to the single use device 204 a peripheral device 206 is desired, the peripheral device adapter 210 is inserted into the capital equipment connector 208 and the single use device connector 212 is inserted into the peripheral device adapter 210. As illustrated in FIG. 4, when a procedure requires more than one peripheral device 206, the peripheral device adapters 210, 210′ may be connected together. As such, each peripheral device adapter 210, 210′ is configured to connect to an additional peripheral device adapter 210, 210′ as well as to a single use device connector 212.

The peripheral device adapter 210 includes a housing having a first end 214 configured to connect to the capital equipment 202 and a second end 216 configured to connect to a single use device 204. In some embodiments, the second end 216 is configured to connect to an over the wire connector that connects with a single use device 204, such as an RF puncturing or perforation device. An over the wire connector is configured to electrically connect with the single use device 204 by receiving a portion of the single use device 204 in a lumen or channel having one or more electrical contacts. As discussed above, the second end 216 is also configured to connect to a first end 214 of an additional peripheral device adapter 210′ as illustrated in FIG. 4.

The peripheral device adapter 210 includes a peripheral device connector 211 that can electrically and mechanically connect with a peripheral device 206. The peripheral device connector 211 allows for removable connection to different types of peripheral devices 206.

The single use device connector 212 includes single use device memory 220. Single use device memory 220 can include any type of memory including, for example, eeprom, flash, SD, MMC, XD, or SDHC. The single use device memory 220 includes data or information for identifying the single use device 204 to the capital equipment 202 when connected as well as information or parameters associated with operation of the single use device 204. For instance, the data or information stored on the single use device memory 220 can include operating parameters regarding the set up and operation of the single use device 204 in combination with the capital equipment 202. The capital equipment 202 is configured to read the parameters to program the controls to be suited for the associated single use device 204. In some embodiments, the capital equipment 202 can be programmed to write to memory segments on the single use device memory device 220 as well as read the single use memory device 220.

Similarly, the peripheral device adapter 210 includes peripheral device adapter memory 218. Peripheral device adapter memory 218 can include any type of memory including, for example, eeprom, flash, SD, MMC, XD, or SDHC. The peripheral device adapter memory 218 includes data or information for identifying the peripheral device 206 to the capital equipment 202 when connected as well as data or information associated with operation of the peripheral device 206. The data or information may include parameters for the functioning or control of the peripheral device 206. Because the capital equipment 202 is configured to communicate with the simplified data structure associated with single use devices, the peripheral device adapter memory 218 includes instructions allowing the conversion of information or data associated with the peripheral device 206 into a simplified data structure similar associated with the single use device 204. The data or information is packaged or formatted as a data structure associated with a single use device 204 to allow for communication with the capital equipment 202 in a simplified manner. In other words, the peripheral device adapter memory 218 includes instructions to reduce complex information or data associated with a peripheral device 206, for example measurements or control parameters, to be packaged into a format representative of a single use device 204 to allow for communication with the capital equipment 202. The peripheral device adapter memory 218 allows for any information or data associated with the peripheral device 206 to be emulated as a single use device 204 for reading by the capital equipment 202.

The capital equipment 202 communicates with the single use device 204 and the peripheral device 206 using a low-level data communication protocol. The low-level data communication protocol can take the form of any low-level data communication protocol including 1-wire, inter-integrated circuit (I2C), CAN, or utilizing parallel-bus architecture. The capital equipment connector 208, peripheral device adapter 210, or single use device connector 212 can be configured as connectors specific for a desired protocol, such as 1-wire, inter-integrated circuit (I2C), CAN, or parallel-bus architecture. The connectors can include various types of connectors such as coaxial, pin connectors, or any male or female type connector.

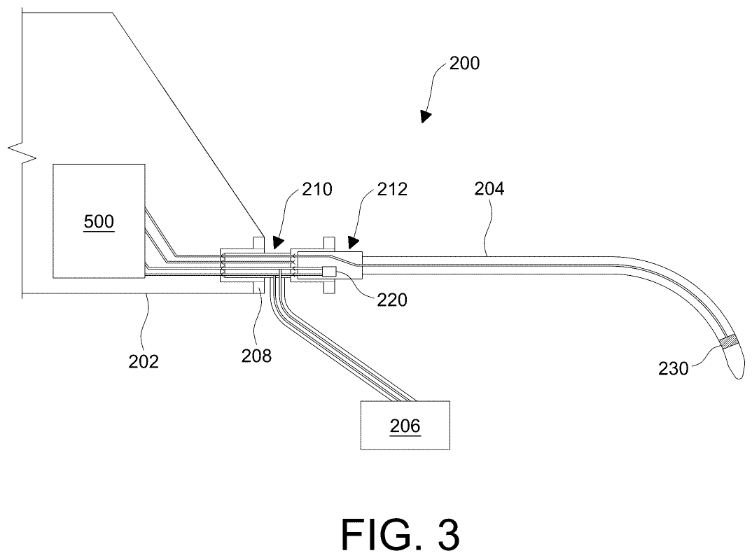

When the system 200 is assembled as illustrated in FIG. 3, the capital equipment 202 is able to transfer information and signals to the peripheral device 206 as well as the single use device 204. Signals from the capital equipment 202 are able to pass through the peripheral device adapter 210 to the single use device 204. The signals can include signals for transferring information to or from the single use device 204, such as information related to physiological parameters or mapping information. Additionally, the signals can include energy for delivery to a patient for therapy. In some aspects, energy is delivered from the capital equipment 202 through the peripheral device adapter 210 to one or more electrodes of a single use device 204 connected to the second end 216 of the peripheral device adapter 210 in order to perforate tissue during a transseptal crossing procedure. In some aspects, energy is delivered from the capital equipment 202 through the peripheral device adapter 210 to one or more electrodes of a single use device 204 connected to the second end 216 of the peripheral device adapter 210 in order to ablate tissue.

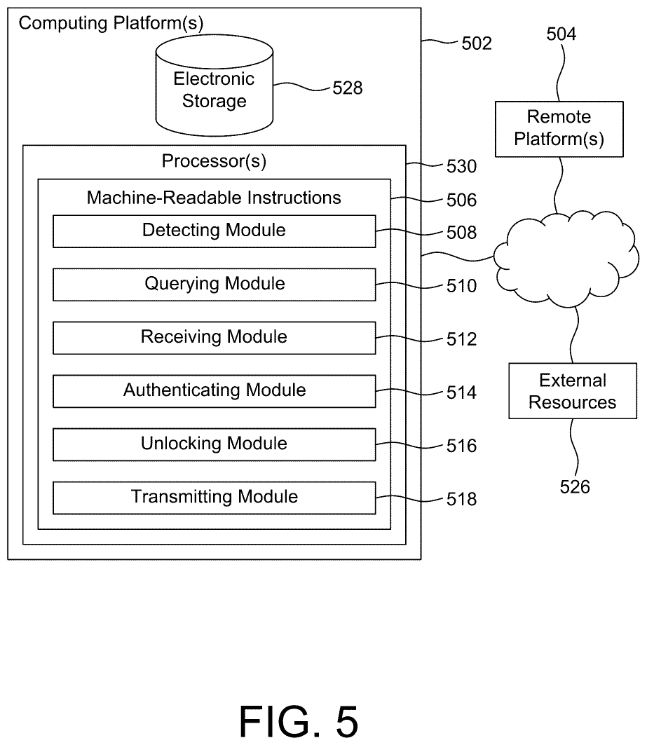

The capital equipment 202 includes one or more computing platforms 502 for controlling communication between the peripheral device 206, the capital equipment 202, and single use device 204. The capital equipment 202 includes a plurality of wires or traces 222 configured to transmit signals or information between the peripheral device 206, single use device 204, and the computing platform(s). As illustrated in FIG. 5, computing platform(s) 502 may be configured to communicate with one or more remote platforms 504 according to a client/server architecture, a peer-to-peer architecture, and/or other architectures. Remote platform(s) 504 may be configured to communicate with other remote platforms via computing platform(s) 502 and/or according to a client/server architecture, a peer-to-peer architecture, and/or other architectures. Users may access the computing platform(s) via remote platform(s) 504.

Computing platform(s) 502 may be configured by machine-readable instructions 506. Machine-readable instructions 506 may include one or more instruction modules. The instruction modules may include computer program modules. The instruction modules may include one or more of detecting module 508, querying module 510, receiving module 512, authenticating module 514, unlocking module 516, transmitting module 518, and/or other instruction modules.

Detecting module 508 is configured to detect the presence of a single use device 204 and a peripheral device 206 connected to the capital equipment 202. The Detecting module 508 is configured to identify when a single use device 204 or peripheral device 206 is connected to the capital equipment 202. The detecting module 508 is configured to access information stored in the peripheral device adapter memory 218 or the single use device memory 220. The detecting module 508 identifies a connected peripheral device 206 as an emulated single use device. That is, the peripheral device adapter memory 218 includes information to allow the peripheral device 206 to be viewed as a single use device by the computing platform 502. The peripheral device adapter memory 218 includes instructions to package data or information associated with the peripheral device 206 into a format representative of a single use device 204. By providing peripheral devices 206 the ability to be viewed as an emulated single use device, the capital equipment 202 can interface with a variety of peripheral devices without specific hardware or software. The detecting module 508 polls the capital equipment connector 208 to view any newly connected devices. After scanning the peripheral device 218, the detecting module 508 identifies an emulated single use device or data packaged in a format representative of a single use device 204.

After identification, the querying module 510 queries the peripheral device 206. The querying module 510 is configured to present a query to the peripheral device 206. The querying module 510 can deliver information to the peripheral device 206, such as a secure key, or request for a key, or other information to the peripheral device 206 that has been identified as an emulated single use device. Alternatively, the querying module 510 can send a challenge to the peripheral device 206 which prompts a response.

Receiving module 512 is configured for receiving a response from the peripheral device. The receiving module 512 identifies a response from the peripheral device 206 in response to the query provided by the querying module 510.

Authenticating module 514 is configured for authenticating the peripheral device 206. The authenticating module 514 verifies the response received by the receiving module 512 to authenticate the peripheral device 206. The authenticating module 514 finalizes a secure connection between the computer platform(s) 502 and the peripheral device 206.

Unlocking module 516 is configured to unlocking one or more pages in the peripheral device adapter memory 218. This allows for unlocking of data within the peripheral device adapter memory 218 and sharing of the data between the peripheral device 206 and the computer platform(s) 502. For example, data associated with a pressure sensor of a peripheral device 206 can be received by the computer platform(s) 502 for use by the capital equipment 202.

Transmitting module 518 is configured to transmitting information, for example data from a sensor, between the connected peripheral device 206 and the capital equipment 202. Transmitting information between the connected peripheral device 206 and the capital equipment 202 includes receiving information from the connected peripheral device 206 and sending information to the connected peripheral device 206. This allows for contents of one or more pages unlocked in the peripheral device adapter memory 218 to be updated dynamically, for example by downloading or uploading information to the one or more pages.

In some implementations, computing platform(s) 502, remote platform(s) 504, and/or external resources 526 may be operatively linked via one or more electronic communication links. For example, such electronic communication links may be established, at least in part, via a network such as the Internet and/or other networks. It will be appreciated that this is not intended to be limiting, and that the scope of this disclosure includes implementations in which computing platform(s) 502, remote platform(s) 504, and/or external resources 526 may be operatively linked via some other communication media.

A given remote platform 504 may include one or more processors configured to execute computer program modules. The computer program modules may be configured to enable an expert or user associated with the given remote platform 504 to interface with the computing platform(s) 502 and/or external resources 526, and/or provide other functionality attributed herein to remote platform(s) 504. By way of non-limiting example, a given remote platform 504 and/or a given computing platform 500 may include one or more of a server, a desktop computer, a laptop computer, a handheld computer, a tablet computing platform, a NetBook, a Smartphone, a gaming console, and/or other computing platforms.

External resources 526 may include sources of information outside of the computing platform(s) 502, external entities participating with the computing platform(s) 502, and/or other resources. In some implementations, some or all of the functionality attributed herein to external resources 526 may be provided by resources included in the computing platform(s) 502.

Computing platform(s) 502 may include electronic storage 528, one or more processors 530, and/or other components. Computing platform(s) 502 may include communication lines, or ports to enable the exchange of information with a network and/or other computing platforms. Illustration of computing platform(s) 502 in FIG. 5 is not intended to be limiting. Computing platform(s) 502 may include a plurality of hardware, software, and/or firmware components operating together to provide the functionality attributed herein to computing platform(s) 502. For example, computing platform(s) 502 may be implemented by a cloud of computing platforms operating together as computing platform(s) 502.

Electronic storage or memory 528 may comprise non-transitory storage media that electronically stores information. The electronic storage media of electronic storage 528 may include one or both of system storage that is provided integrally (i.e., substantially non-removable) with computing platform(s) 502 and/or removable storage that is removably connectable to computing platform(s) 502 via, for example, a port (e.g., a USB port, a firewire port, etc.) or a drive (e.g., a disk drive, etc.). Electronic storage 528 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 528 may include one or more virtual storage resources (e.g., cloud storage, a virtual private network, and/or other virtual storage resources). Electronic storage 528 may store instructions, software algorithms, information determined by processor(s) 530, information received from computing platform(s) 502, information received from remote platform(s) 504, and/or other information that enables computing platform(s) 502 to function as described herein.

Processor(s) 530 may be configured to provide information processing capabilities in computing platform(s) 502 and are configured to perform operations as instructed. As such, processor(s) 530 may include one or more of a digital processor, an analog processor, a digital circuit designed to process information, an analog circuit designed to process information, a state machine, and/or other mechanisms for electronically processing information. Although processor(s) 530 is shown in FIG. 5 as a single entity, this is for illustrative purposes only. In some implementations, processor(s) 530 may include a plurality of processing units. These processing units may be physically located within the same device, or processor(s) 530 may represent processing functionality of a plurality of devices operating in coordination. Processor(s) 530 may be configured to execute modules 508, 510, 512, 514, 516, and/or 518, and/or other modules. Processor(s) 530 may be configured to execute modules 508, 510, 512, 514, 516, and/or 518, and/or other modules by software; hardware; firmware; some combination of software, hardware, and/or firmware; and/or other mechanisms for configuring processing capabilities on processor(s) 530. As used herein, the term “module” may refer to any component or set of components that perform the functionality attributed to the module. This may include one or more physical processors during execution of processor readable instructions, the processor readable instructions, circuitry, hardware, storage media, or any other components.

It should be appreciated that although modules 508, 510, 512, 514, 516, and/or 518 are illustrated in FIG. 5 as being implemented within a single processing unit, in implementations in which processor(s) 530 includes multiple processing units, one or more of modules 508, 510, 512, 514, 516, and/or 518 may be implemented remotely from the other modules. The description of the functionality provided by the different modules 508, 510, 512, 514, 516, and/or 518 described below is for illustrative purposes, and is not intended to be limiting, as any of modules 508, 510, 512, 514, 516, and/or 518 may provide more or less functionality than is described. For example, one or more of modules 508, 510, 512, 514, 516, and/or 518 may be eliminated, and some or all of its functionality may be provided by other ones of modules 508, 510, 512, 514, 516, and/or 518. As another example, processor(s) 530 may be configured to execute one or more additional modules that may perform some or all of the functionality attributed below to one of modules 508, 510, 512, 514, 516, and/or 518.

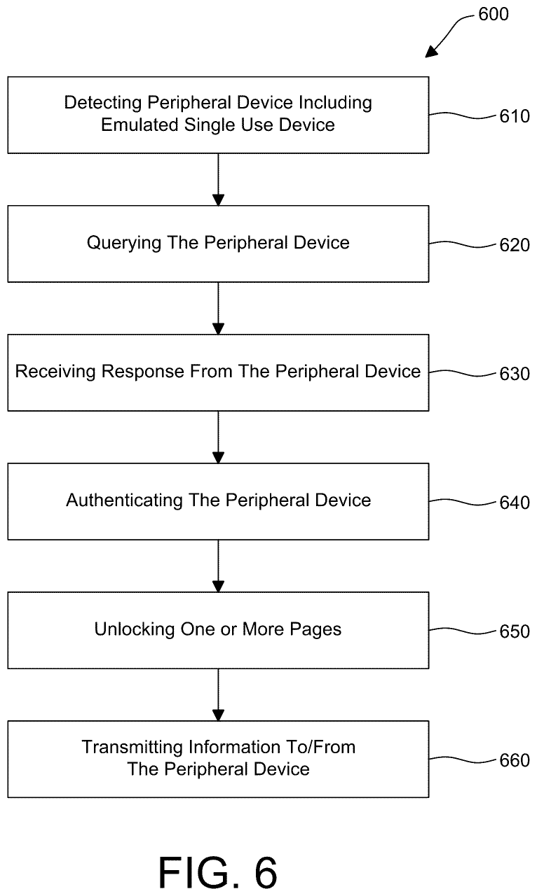

FIG. 6 illustrates a method 600 for connecting peripheral devices to capital equipment in order to allow communication between the capital equipment and the peripheral devices, in accordance with an embodiment of the disclosure. The operations of method 600 presented below are intended to be illustrative. In some implementations, method 600 may be accomplished with one or more additional operations not described, and/or without one or more of the operations discussed. Additionally, the order in which the operations of method 600 are illustrated in FIG. 6 and described below is not intended to be limiting.

In some implementations, method 600 may be implemented in the capital equipment 202 using one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 600 in response to instructions stored electronically on an electronic storage medium. The one or more processing devices may include one or more devices configured through hardware, firmware, and/or software to be specifically designed for execution of one or more of the operations of method 600.

An operation 610 includes detecting a peripheral device connected to a piece capital equipment, wherein the peripheral device includes an emulated single use device. Operation 610 may be performed by one or more hardware processors configured by machine-readable instructions including a module that is the same as or similar to detecting module 508, in accordance with one or more implementations.

An operation 620 includes querying the peripheral device. Operation 620 may be performed by one or more hardware processors configured by machine-readable instructions including a module that is the same as or similar to querying module 510, in accordance with one or more implementations.

An operation 630 includes receiving a response from the peripheral device. Operation 630 may be performed by one or more hardware processors configured by machine-readable instructions including a module that is the same as or similar to receiving module 512, in accordance with one or more implementations.

An operation 640 includes authenticating the peripheral device. Operation 640 may be performed by one or more hardware processors configured by machine-readable instructions including a module that is the same as or similar to authenticating module 514, in accordance with one or more implementations.

An operation 650 includes unlocking one or more pages in the memory of the peripheral device. Operation 650 may be performed by one or more hardware processors configured by machine-readable instructions including a module that is the same as or similar to unlocking module 516, in accordance with one or more implementations.

An operation 660 includes transmitting information between the connected peripheral device and the capital equipment. Operation 660 may be performed by one or more hardware processors configured by machine-readable instructions including a module that is the same as or similar to transmitting module 518, in accordance with one or more implementations.

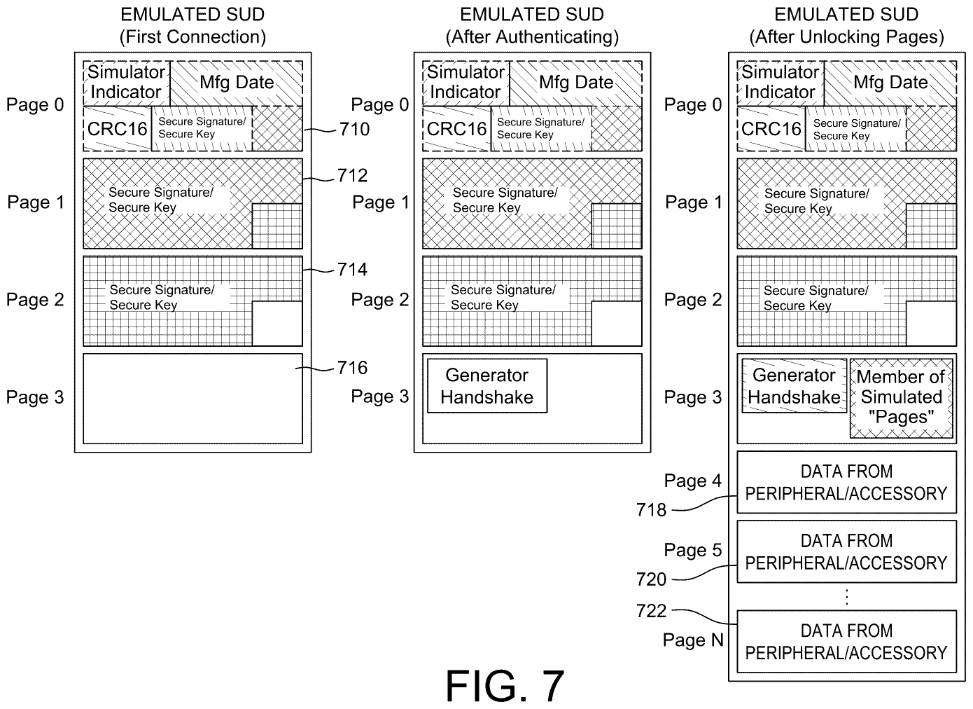

FIG. 7 illustrates one or more simulated pages located in a peripheral device adapter memory, in accordance with an embodiment of the disclosure. Upon initial connection of a peripheral device 206 to a piece of capital equipment 202, the computing device 502 identifies an emulated single use device and reads page zero 710, page one 712, and page two 714 from the peripheral device adapter memory 218. Pages zero through two 710, 712, 714 contain information related to manufacturer, type, and other data related to the kind of peripheral device. Page three 716 remains empty until the authentication module 514 authenticates the emulated single use device. Page three 716 can include data associated with the authentication and creation of a secure connection between the capital equipment 202 and the peripheral device 206. Upon the creation of a secure connection between the capital equipment 202 and a connected peripheral device 206, one or more additional pages 718, 720, 722 open as needed to share data or information with the capital equipment 202. The data or information can be encoded to allow the capital equipment 202 to understand the formatting of data or information in the one or more additional pages 718, 720, 722. Each of the one or more additional pages 718, 720, 722 can be formatted to include specific information or data for each peripheral device 206 connected to the capital equipment 202. It is understood that one type of peripheral device 206 may only require one page for management of the data or information while a different type of peripheral device 206 may require a plurality of pages for management of the data or information.

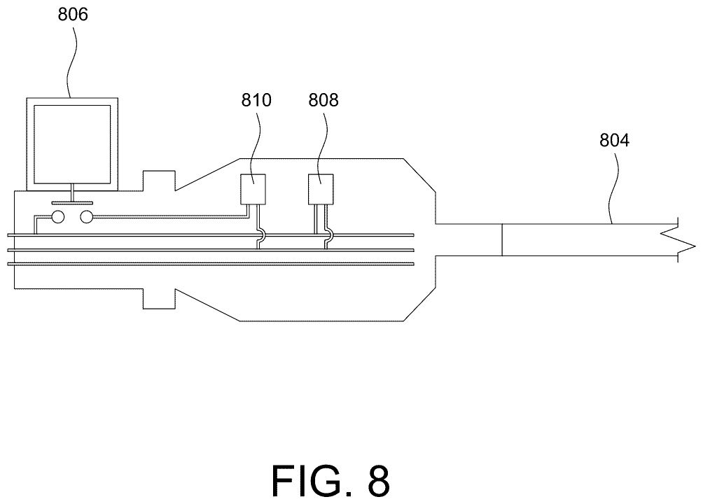

FIG. 8 is a schematic representation of a single use device connector 804 having a push button switch 806, in accordance with an embodiment of the disclosure. In addition to single use device memory 808, the single use device connector 804 includes push button memory 810. Upon activation of the push button switch 806, one or more of the detecting module 508, receiving module 510, authenticating module 514 and unlocking module 516, identifies the information stored on the push button memory 810 indicating activation of the push button switch 806.

Activation of the push button switch 806 can allow the single use device 204 associated with the single use device connector 804 to accomplish an alternate functionality. For example, the single use device memory 808 may include information such as one or more keys or codes to allow for authenticating of a treatment plan delivered by the single use device 204. The information stored in the single use device memory 808 can also include specific instructions for a desired treatment plan. The instructions may dictate specific voltages, frequencies, current densities, and durations to be delivered by an electrode associated with the single use device 204. Upon actuation of the push button switch 806, the capital equipment 202 accesses the push button memory 810 which can include information, that when communicated to the capital equipment 202, can change or modify the treatment plan. Modification of the treatment plan includes changing a sequence, duration, voltage, or frequency of RF energy delivery. In some aspects, the information associated with the push button memory 81 may be communicated to an additional control system, for example an attached peripheral device 206. Modification of the treatment plan can include receiving control signals from an additional control system.

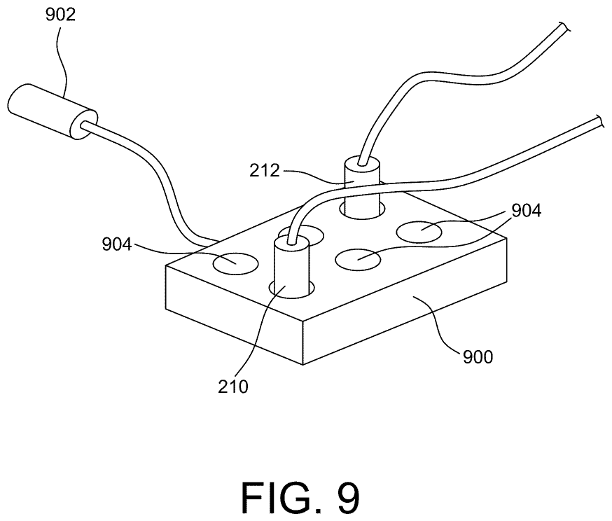

In an alternative arrangement, the system 200 can include an expansion block 900 as illustrated in FIG. 9. The expansion block 900 includes an expansion block connector 902 that is configured to mechanically and electrically connect with the capital equipment connector 208. A plurality of openings or slots 904 are arranged throughout the expansion block 900. The plurality of openings or slots 904 are configured to receive multiple peripheral device adapters 210 or single use device connectors 212. The expansion block 900 allows for the connection of multiple peripheral devices 206 and/or multiple single use devices 204 to the capital equipment 202 simultaneously. In some embodiments, the expansion block 900 may be configured to connect with only one single use device 204 and a plurality of peripheral devices 206. In such an arrangement, one of the plurality of openings or slots 904 may be designated for connection with the single use device 204. For example, one of the plurality of openings or slots 904 may include coloration or indicia to identify the opening or slot for use with a single use device 204. In some embodiments, a light or other status indicator adjacent the plurality of opening or slots may be used to indicate successful connection with the capital equipment upon receipt of a peripheral device adapter 210 or single use device connector 212. Communication between any connected peripheral device 206 or single use device 204 and the capital equipment 202 follows the protocol as discussed above.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A system for performing a medical procedure using a piece of capital equipment, the system comprising:
a peripheral device adapter, the adapter comprising:
a housing having a first end configured to connect to the piece of capital equipment and a second end configured to connect to a single use device;
a connector configured to removably connect to a peripheral device; and
a peripheral device adapter memory device for storing instructions, wherein the instructions are configured to package data or information associated with the peripheral device into a format representative of a single use device to allow for communication with the piece of capital equipment; and
one or more processors configured for:
detecting a peripheral device connected to the peripheral device adapter when the peripheral device adapter is connected to the piece of capital equipment;
querying the detected connected peripheral device;
receiving a response from the connected peripheral device; and
authenticating the connected peripheral device.

2. The system of claim 1, wherein the peripheral device includes one or more sensor, therapy controller, monitor, or therapy device.

3. The system of claim 1, wherein the piece of capital equipment includes a control system, power or energy generator, ablation system, therapy controller, or console.

4. The system of claim 1, wherein the single use device includes one or more lead, perforation device, electrode, catheter, sheath, guidewire, or dilator.

5. The system of claim 1, wherein energy is delivered through the peripheral device adapter to one or more electrodes of the single use device connected to the second end of the peripheral device adapter in order to perforate tissue during a transseptal crossing procedure.

6. The system of claim 1, wherein energy is delivered through the peripheral device adapter to one or more electrodes of the single use device connected to the second end of the peripheral device adapter in order to ablate tissue.

7. The system of claim 1, wherein the peripheral device adapter is configured for 1-wire, I2C, CAN, parallel-bus architecture, or low-level data communication protocol.

8. The system of claim 1, wherein the peripheral device adapter memory is removable.

9. The system of claim 1, wherein the peripheral device adapter memory includes eeprom, flash, SD, MMC, XD, or SDHC.

10. The system of claim 1, wherein the peripheral device adapter is configured to connect to an additional peripheral device adapter.

11. The system of claim 1, wherein the second end configured to connect to a single use device includes an over the wire connector.

12. The system of claim 1, wherein the peripheral device adapter allows for energy to pass from the piece of capital equipment to the single use device for delivery to a patient for therapy.

13. The system of claim 1, wherein the one or more processors is further configured for:
unlocking one or more pages in the peripheral device adapter memory; and
transmitting information between the peripheral device and the piece of capital equipment.

14. A method for communicating between a peripheral device and a piece of capital equipment, the method comprising:
detecting the peripheral device connected to the piece of capital equipment, wherein the peripheral device includes data or information packaged into a format representative of a single use device;
querying the peripheral device;
receiving a response from the peripheral device;
authenticating the peripheral device;
unlocking one or more pages in a memory of the peripheral device; and
transmitting information between the connected peripheral device and the piece of capital equipment.

15. The method of claim 14, wherein transmitting information between the connected peripheral device and the piece of capital equipment includes receiving information from the connected peripheral device and sending information to the connected peripheral device.

16. The method of claim 14, wherein the one or more pages includes access to data associated with the connected peripheral device.

17. The method of claim 14, wherein contents of the one or more pages can be updated dynamically.

18. The method of claim 14, further comprising electrically connecting a single use device to the piece of capital equipment.

19. The method of claim 18, further comprising sending signals from the piece of capital equipment to the single use device though an adapter electrically connecting the peripheral device, the capital equipment, and the single use device.

20. The method of claim 19, further comprising:
identifying data indicating activation of a switch on the single use device; and
modifying the signals.
